(19) United States
(12) Patent Application Publication
(10) Pub. No.: US 2009/0041764 A1
(43) Pub. Date: Feb. 12, 2009

(54) Methods for the treatment of muscular dystrophy associated with dysferlin-deficiency

(75) Inventors: Simone Spuler, Berlin (DE); Russell P. Rother, Prospect, CT (US); Katrin Wenzel, Bernau (DE)

Correspondence Address: ROPES & GRAY LLP, PATENT DOCKETING 39/41, ONE INTERNATIONAL PLACE, BOSTON, MA, 02110-2624, US

(73) Assignee: Alexion Pharmaceuticals, Inc., Cheshire, CT (US)

(21) Appl. No.: 11/975,603

(22) Filed: Oct. 19, 2007

Related U.S. Application Data
(60) Provisional application No. 60/853,213, filed on Oct. 20, 2006.

(51) Int. Cl. A61K 39/395; A61K 38/00; A61K 31/7088

(52) U.S. Cl. 424/133.1; 514/2; 424/130.1; 514/44; 424/141.1

(57) ABSTRACT

The use of therapeutics capable of inhibiting complement such as an anti-C5 antibody to treat muscular dystrophy associated with dysferlin-deficiency is disclosed.

### CROSS REFERENCE TO RELATED APPLICATIONS

This application claims the benefit of U.S. Provisional Application Ser. No. 60/853,213, filed on Oct. 20, 2006, the entire contents of which is incorporated herein by reference.

### FIELD OF THE INVENTION

The present invention relates to methods for the treatment of muscular dystrophy associated with dysferlin-deficiency. In specific embodiments, the invention relates to the use of antibodies capable of inhibiting complement as therapeutic agents to treat muscular dystrophy associated with dysferlin-deficiency.

### BACKGROUND OF THE INVENTION

Muscular dystrophy represents a family of inherited diseases of the muscles. To date, there is no known treatment, medicine, or surgery that will cure muscular dystrophy, or stop the muscles from weakening. There has thus been a long felt need for new approaches and better methods to treat muscular dystrophy, including muscular dystrophy associated with dysferlin-deficiency.

### SUMMARY OF THE INVENTION

Accordingly, the disclosure provides methods and compositions useful for treating muscular dystrophy, particularly muscular dystrophy associated with dysferlin-deficiency.

In certain embodiments, the disclosure provides a method of treating muscular dystrophy associated with dysferlin-deficiency in a mammal comprising administering to said mammal a therapeutically effective amount of an agent (e.g., an antibody or fragment thereof) that inhibits complement, such as for example by inhibiting the formation of the membrane attack complex (MAC). In specific embodiments, the agent is an antibody that comprises anti-C5 antibody, such as for example an antibody that binds CS and prevents the cleavage of C5 into C5a and C5b. In certain embodiments, the mammal is a human.

In certain embodiments, the antibody is a whole antibody or an antibody fragment. In certain embodiments, the whole antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, and an F(ab′)₂. In certain embodiments, the antibody is pexelizumab. In certain embodiments, the antibody is eculizumab.

In certain embodiments, the agent is administered chronically to said mammal. In certain embodiments, said mammal receives a one-time administration or multiple administrations of the agent during a limited time period such as a week, a month, a year or longer.

In certain embodiments, the agent is administered systemically to said mammal. In certain embodiments, the agent is administered locally to said mammal.

In certain embodiments, the agent is administered in combination with another therapeutic agent to said mammal.

In certain embodiments, the therapeutic agent comprises an amino acid sequence of greater than 90% sequence identity to the amino acid sequence of a soluble portion of a naturally occurring CD55 protein. In certain embodiments, the therapeutic agent comprises a polynucleotide sequence of greater than 90% sequence identity to the nucleotide sequence of a naturally occurring CD55 mRNA.

In certain embodiments, the disclosure provides the use of an anti-C5 antibody in the manufacture of a medicament or medicament package for the treatment of muscular dystrophy associated with dysferlin-deficiency in a mammal.

In certain embodiments, the disclosure provides the use of a compound with CD55 activity in the manufacture of a medicament or medicament package for the treatment of muscular dystrophy associated with dysferlin-deficiency in a mammal.

In certain embodiments, the disclosure provides a method of limiting the generation of necrotic muscle fibers in muscular dystrophy associated with dysferlin-deficiency in a mammal comprising administering to said mammal a therapeutically effective amount of a complement inhibitor, e.g., an anti-C5 antibody. In certain embodiments, the disclosure provides a method of reducing necrosis of muscle fibers in muscular dystrophy associated with dysferlin-deficiency in a mammal comprising administering to said mammal a therapeutically effective amount of a complement inhibitor, e.g., an anti-C5 antibody.

In certain embodiments, the disclosure provides a method of limiting the generation of necrotic muscle fibers in muscular dystrophy associated with dysferlin-deficiency in a mammal comprising administering to said mammal a therapeutically effective amount of a compound with CD55 activity. In certain embodiments, the disclosure provides a method of reducing necrosis of muscle fibers in muscular dystrophy associated with dysferlin-deficiency in a mammal comprising administering to said mammal a therapeutically effective amount of a compound with CD55 activity.

In certain embodiments, the disclosure provides a use of an anti-C5 antibody in the manufacture of a medicament or medicament package for limiting the generation of necrotic muscle fibers in muscular dystrophy associated with dysferlin-deficiency in a mammal. In certain embodiments, the disclosure provides a use of an anti-C5 antibody in the manufacture of a medicament or medicament package for reducing necrosis of muscle fibers in muscular dystrophy associated with dysferlin-deficiency in a mammal.

The invention contemplates combinations of any of the foregoing aspects and embodiments of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. TaqMan RT-PCR amplification plots of DAF/CD55. A and C, DAF1 (A) and DAF2 (C) expression in skeletal muscle (M. quadriceps) of a 30-wk-old SJL/J mouse compared with a C57BL/6 control mouse of the same age (Ctrl). E, DAF/CD55 expression in skeletal muscle of a patient with LGMD2B (patient 4, Table III) compared with a healthy control. Housekeeping genes porphobilinogen deaminase (B and D) and β₂-microglobulin (F) were used as internal standards.

FIG. 2. DAF/CD55 protein expression in dysferlin deficiency. Immunofluorescence staining using anti-DAF/CD55 Abs. A-D, Murine skeletal muscle. A-D have the same scale as indicated in B. E and F, Murine cardiac muscle. E and F have the same scale as indicated in F. G and H, Human skeletal muscle. G and H have the same scale as indicated in H. A, SJL/J, wk 28; B, A/J wk 16; C, Dysf^{-l-}, wk 16; D, C57BL/6; E, SJL/J; F, C57BL/6; G, LGMD2B patient 2 (Table III); H, control skeletal muscle without detectable neuromuscular disorder.

FIG. 3. Complement lysis assay and binding of C5b9-MAC to nonnecrotic muscle cells. A, Quantification of PI uptake of myotubes after exposure to complement (ratio of PI-positive cells after exposure to complement to Veronal buffer control). n, number of wells counted. Normal human (B and C) and dysferlin-deficient (D and E) human myoblasts after exposure to complement (B and D) and after preincubation with anti-CD55 Ab and subsequent exposure to complement (C and E). F and G, Serial sections of quadriceps muscle in LGMD2B (patient 1), demonstrating dystrophic changes with increase in connective tissue and pathological variation in fiber size (Gomori-TriChrome stain). There was sarcolemmal expression of C5b9-MAC on nonnecrotic muscle fibers. Staining was performed with anti-C5b9 mAb and Cy3-labeled donkey anti-mouse Ab.

FIG. 4. Expression of regulatory factors in skeletal muscle of dysferlin-deficient patients and SJL/J mice (aged 20-30 wk). A, Unpooled TaqMan analysis of myostatin, SMAD3, SMAD4, CARP, and EGR1 (only human). The y-axis demonstrates the fold change compared with healthy individuals and C57BL/6 mice, respectively. B and C, Double-immunofluorescent staining of SMAD2 protein (FITC) and nuclear membrane with anti-lamin A/C mAb (Cy3) on dysferlin-deficient (patient 4, Table III; B) and normal (C) human skeletal muscle.

FIG. 5. Anti-C5 antibody alleviates dysferlin-deficient muscular dystrophy in mice. A, Natural course of quadriceps pathology in untreated SJL/J mice. Each group consisted of 3-6 animals. An increase in the percentage of necrotic fibers was observed after week 20. B, Gomori trichrome stain of quadriceps obtained from SJL/J mouse after 4 weeks of anti-C5 monoclonal antibody treatment. C, Gomori trichrome stain of quadriceps obtained from SJL/J mouse after 4 weeks of IgG1 isotype control antibody treatment. D, Number of muscle samples with <1% necrotic fibers (“normal”) as compared to 1% or more necrotic fibers (“diseased”) after 4 weeks of treatment with anti-C5 monoclonal antibody (left), albumin (middle), or isotype control antibody (right). Statistical analysis was performed using the Chi-square test.

### DETAILED DESCRIPTION OF THE INVENTION

### Overview

The present invention relates, in part, to the discovery that CD55 is down-regulated in the skeletal muscle of dysferlin-deficient mice or human patients suffering from LGMD2B. Accordingly, methods and compositions are provided for treating muscular dystrophy, particularly muscular dystrophy associated with dysferlin deficiency. The term “treating” includes prophylactic and/or therapeutic treatments. The term “prophylactic or therapeutic” treatment is art-recognized and includes administration to the host of one or more of the subject therapeutic agents or pharmaceutical compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

In certain embodiments, the methods and compositions of the disclosure employ a therapeutic agent that can inhibit complement activity, such as for example, by preventing the formation of MAC; in specific embodiments, such a therapeutic agent may comprise an antibody that binds to C5 and inhibits C5 activity (for example, by preventing the cleavage of C5 into C5a and C5b).

In previous investigations, activation of the complement cascade has been identified on the surface of nonnecrotic muscle fibers in some patients with LGMD (Spuler and Engel. 1998. Neurology 50:41-46) and, in particular, in dysferlinopathies (Selcen et al., 2001. Neurology 56:1472-1481). Deposition of MAC on nonnecrotic muscle fibers in muscular dystrophies was surprising, in particular because this mechanism does not play a role in inflammatory muscle diseases (Spuler and Engel. 1998. Neurology 50:41-46). The complement system consists of >30 plasma and cell surface proteins. It is activated by three different pathways, named classical, alternative, and lectin pathway, respectively (Walport, M. J. 2001. N. Engl. J. Med. 344:1140-1144; Walport, M. J. 2001. N. Engl. J. Med. 344:1058-1066). All pathways require the proteolytic cleavage of C3, followed by the last phase of the complement cascade that leads to the formation of the C5b-9 MAC. To provide an immediate defense against infection, there is a constant low level of C3 activation in the alternative pathway, a background “tick-over” (Pangburn and Muller-Eberhard. 1984. Springer Semin. Immunopathol. 7:163-192). To prevent uncontrolled rapid amplification of the complement cascade and complement-mediated damage of self, numerous soluble and membrane-bound complement inhibitory and regulatory proteins have evolved. Among the membrane-bound inhibitors are decay-accelerating factor (DAF/CD55), membrane cofactor protein (CD46), and CD59 (Morgan, B. P. 1999. Crit. Rev. Immunol. 19:173-198).

### The Complement System

The complement system acts in conjunction with other immunological systems of the body to defend against intrusion of cellular and viral pathogens. There are at least 25 complement proteins, which are found as a complex collection of plasma proteins and membrane cofactors. The plasma proteins (which are also found in most other body fluids, such as lymph, bone marrow, synovial fluid, and cerebrospinal fluid) make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade progresses via the classical pathway or the alternative pathway. These pathways share many components and, while they differ in their early steps, both converge and share the same terminal complement components responsible for the destruction of target cells and viruses.

The classical complement pathway is typically initiated by antibody recognition of and binding to an antigenic site on a target cell. This surface bound antibody subsequently reacts with the first component of complement, C1. The C1 thus bound undergoes a set of autocatalytic reactions that result in, inter alia, the induction of C1 proteolytic activity acting on complement components C2 and C4.

This activated C1 cleaves C2 and C4 into C2a, C2b, C4a, and C4b. The function of C2b is poorly understood. C2a and C4b combine to form the C4b,2a complex, which is an active protease known as classical C3 convertase. C4b,2a acts to cleave C3 into C3a and C3b. C3a and C4a are both relatively weak anaphylatoxins that may induce degranulation of mast cells, resulting in the release of histamine and other mediators of inflammation.

C3b has multiple functions. As opsonin, it binds to bacteria, viruses and 25 other cells and particles and tags them for removal from the circulation. C3b can also form a complex with C4b,C2a to produce C4b,2a,3b, or classical C5 convertase, which cleaves C5 into C5a (another anaphylatoxin) and C5b. Alternative C5 convertase is C3b,Bb,C3b and performs the same function. C5b combines with C6 yielding C5b,6, and this complex combines with C7 to form the ternary complex C5b,6,7. The C5b,6,7 complex binds C8 at the surface of a cell membrane. Upon binding of C9, the complete membrane attack complex (MAC) is formed (C5b-9) which mediates the lysis of foreign cells, microorganisms, and viruses.

Decay accelerating factor (DAF) (CD55) (NM_{—}000574) can bind C4b and C3b dissociating the C3 and C5 convertases in both the classical and alternative pathways (Makrides, Pharmacol Rev. 1998 March ; 50(1):59-87). Soluble versions of DAF (sDAF) have been shown to inhibit complement activation (Christiansen et al., Eur J Immunol. 1996 March; 26(3):578-85; and Moran et al., J Immunol. 1992 Sep. 1;149(5): 1736-43).

CD55 is also a known agonist of CD97. CD97 is a seven-span transmembrane protein that is expressed by leukocytes early after activation. CD97-CD55 interactions play a role in cellular activation, migration, and adhesion under inflammatory conditions, and are involved in the inflammatory process in multiple sclerosis (Visser et al., J. Neuroimmunol. 132:156-163 (2002); and Hamann et al., J Exp Med. 1996 Sep. 1; 184(3):1185-9). Soluble versions of CD55 may prevent inflammation.

Further discussions of the classical complement pathway, as well as a detailed description of the alternative pathway of complement activation, can be found in numerous publications including, for example, Muller-Eberhard, Annu Rev Biochem. 1988;57:321-47.

### Muscular Dystrophy

Muscular dystrophy represents a family of inherited diseases of the muscles. The following are the most common symptoms of muscular dystrophy. Symptoms may include: clumsy movement, difficulty climbing stairs, frequently trips and falls, unable to jump or hop normally, tip toe walking, leg pain, facial weakness, inability to close eyes or whistle, and shoulder and arm weakness.

Some forms affect children (e.g., Duchenne dystrophy) and are lethal within two to three decades. Other forms present in adult life and are more slowly progressive. The genes for several dystrophies have been identified, including Duchenne dystrophy (caused by mutations in the dystrophin gene) and the teenage and adult onset Miyoshi dystrophy or its variant, limb girdle dystrophy 2B or LGMD-2B (caused by mutations in the dysferlin gene). These are “loss of function” mutations that prevent expression of the relevant protein in muscle and thereby cause muscle dysfunction.

Dysferlin is a 230-kDa membrane-spanning protein consisting of a single C-terminal transmembrane domain and six C2 domains (Anderson et al. 1999. Hum. Mol. Genet. 8:855-861). In normal muscle, sarcolemma injuries lead to accumulation of dysferlin-enriched membrane patches and resealing of the membrane in the presence of Ca²⁺. Dysferlin deficiency results in defective membrane repair mechanisms (Bansal et al., 2003. Nature 423:168-172; Lennon et al., 2003. J. Biol. Chem. 278:50466-50473). An impaired interaction between dysferlin and annexins A1 and A2 has been discussed as a possible mechanism (Lennon et al., 2003. J. Biol. Chem. 278:50466-5047). Although dysferlin is expressed in human skeletal and cardiac muscles (Anderson et al., 1999. Hum. Mol. Genet. 8:855-861), mutations in the encoding gene (DYSF) lead only to skeletal muscle phenotypes without myocardial involvement, namely limb girdle muscular dystrophy 2B (LGMD2B) and Miyoshi myopathy (Liu et al., 1998. Nat. Genet. 20:31-36).

Several mouse models exist with mutations in Dysf(Bittner et al., 1999. Nat. Genet. 23:141-142). The SJL/J mouse harbors a splice site mutation that results in a deletion corresponding to human exon 45 (Vafiadaki et al., 2001. NeuroReport 12:625-629). SJL/J mice have long served as a model for autoimmune diseases, such as experimental allergic encephalomyelitis and myositis. The development of lymphomas is typically observed in older age. Therefore, it has been discussed whether other genetic disorders, apart from dysferlin deficiency, might play a role in SJL/J, and more defined models were engineered (Kostek et al., 2002. Am. J. Pathol. 160:833-839; Matsubara et al., 2001. J. Neuroimmunol. 119:223-230). A mouse with a 12-kb deletion at the 3′ end leading to complete loss of dysferlin was designed. In this study the observation of a defective membrane repair mechanism in dysferlin deficiency has been made (Bansal et al., 2003. Nature 423:168-172). The A/J mouse has a unique ETn retrotransposon insertion within intron 4 (Ho et al., 2004. Hum. Mol. Genet. 13:1999-2010). For another targeted disruption of dysferlin, the highly conserved C2E domain was replaced by a neomycin gene, resulting in a Dysf^{-l-} mouse (Ho et al., 2004. Hum. Mol. Genet. 13:1999-2010). All these mice develop progressive muscular dystrophy after 2 mo of age. Interestingly, all mice also display different degrees of inflammatory changes in skeletal muscle.

### Current Therapies

To date, there is no known treatment, medicine, or surgery that will cure muscular dystrophy, or stop the muscles from weakening. The goal of treatment is to prevent deformity and allow the child to function as independently as possible. Since muscular dystrophy is a life-long condition that is not correctable, management includes focusing on preventing or minimizing deformities and maximizing the child's functional ability at home and in the community.

Clinically, many patients with a muscular dystrophy show improvement with prednisone treatment, although they will not be cured (Fenichel et al., Arch Neurol. 1991 June; 48(6):575-9; Griggs et al., Arch Neurol. 1991 April; 48(4):383-8). Dysferlin-deficiency was recently recognized as a cause of late-onset dystrophy with substantial inflammation in muscle. Corticosteroid usage by these patients may result in nonrecoverable loss of strength. There has thus been a long felt need for new approaches and better methods to control muscular dystrophy associated with dysferlin-deficiency.

Khurana and Davies reviewed the various pharmaceutical strategies for muscular dystrophy to date. Nature Reviews 2:379-390 (May 2003). Current therapeutic approaches generally utilize drugs or molecules in an attempt to improve the phenotype by, for example, decreasing inflammation, improving calcium homeostasis, upregulating compensatory protein such as utrophin, increasing muscle progenitor proliferation or commitment, and increasing muscle strength. Specific agents that have been used in patients or in clinical or preclinical studies include corticosteroids, calcium ionophores and/or blockers of the sarcoplasmic reticulum calcium reuptake pump, mast-cell stabilizers such as cromoglycate, clenbutarol (a non-steroid b2 adrenoreceptor agonist), creatine or creatine monohydrate, and gentamicin.

### Methods and Compositions

As discussed above, the present invention relates to a method for treating muscular dystrophy associated with dysferlin-deficiency by the administration of an agent capable of inhibiting complement (for example, by inhibiting the formation of MAC) to a patient in need of such treatment. In particular embodiments, the agent inhibits the formation of the MAC by inhibiting the cleavage of C5 into C5a and C5b or the formation of C3 and/or C5 convertases.

In certain embodiments, a complement inhibitor may be a small molecule (up to 6,000 Da in molecular weight), a nucleic acid or nucleic acid analog, a peptidomimetic, or a macromolecule that is not a nucleic acid or a protein. These agents include, but are not limited to, small organic molecules, RNA aptamers, L-RNA aptamers, Spiegelmers, antisense compounds, double stranded RNA, small interfering RNA, locked nucleic acid inhibitors, and peptide nucleic acid inhibitors.

In certain embodiments, complement inhibitor may be an antibody capable of inhibiting complement, such as an antibody that can block the formation of MAC. For example, an antibody complement inhibitor may include an anti-C5 antibody. Such anti-C5 antibodies may directly interact with C5 or C5b, so as to inhibit the formation of and/or physiologic function of C5b. Furthermore, they may inhibit the formation of C5a.

The concentration and/or physiologic activity of C5a and C5b in a body fluid can be measured by methods well known in the art. For C5a such methods include chemotaxis assays, RIAs, or ELISAs (see, for example, Ward and Zvaifler, J Clin Invest. 1971 March; 50(3):606-16; Wurzner et al., Complement Inflamm. 8:328-340, 1991). For C5b, hemolytic assays or assays for soluble C5b-9 as discussed herein can be used. Other assays known in the art can also be used. Using assays of these or other suitable types, candidate antibodies capable of inhibiting complement such as anti-C5 antibodies, now known or subsequently identified, can be screened in order to 1) identify compounds that are useful in the practice of the invention and 2) determine the appropriate dosage levels of such compounds.

An antibody capable of inhibiting complement such as an anti-C5 antibody affecting C5b and/or C5a is preferably used at concentrations providing substantial reduction (i.e., reduction by at least about 25% as compared to that in the absence of the anti-C5 antibody) in the C5b and/or C5a levels present in at least one blood-derived fluid of the patient following activation of complement within the fluid. In the case of C5b, such concentrations can be conveniently determined by measuring the cell-lysing ability (e.g., hemolytic activity) of complement present in the fluid or the levels of soluble C5b-9 present in the fluid. Accordingly, a specific concentration for an antibody that affects C5b is one that results in a substantial reduction (i.e., a reduction by at least about 25%) in the cell-lysing ability of the complement present in at least one of the patient's blood-derived fluids. Reductions of the cell-lysing ability of complement present in the patient's body fluids can be measured by methods well known in the art such as, for example, by a conventional hemolytic assay such as the hemolysis assay described by Kabat and Mayer (eds), “Experimental Immunochemistry, 2d Edition”, 135-240, Springfield, Ill., C C Thomas (1961), pages 135-139, or a conventional variation of that assay such as the chicken erythrocyte hemolysis method described below.

Specific antibodies capable of inhibiting complement such as an anti-C5 antibody are relatively specific, and preferably do not block the functions of early complement components. In particular, such specific agents preferably will not substantially impair the opsonization functions associated with complement component C3b, which functions provide a means for clearance of foreign particles and substances from the body.

C3b is generated by the cleavage of C3, which is carried out by classical and/or alternative C3 convertases, and results in the generation of both C3a and C3b. Therefore, in order not to impair the opsonization functions associated with C3b, specific antibodies capable of inhibiting complement such as an anti-C5 antibody do not substantially interfere with the cleavage of complement component C3 in a body fluid of the patient (e.g., serum) into C3a and C3b. Such interference with the cleavage of C3 can be detected by measuring body fluid levels of C3a and/or C3b, which are produced in equimolar ratios by the actions of the C3 convertases.

In practice, the quantitative measurement of such cleavage is generally more accurate when carried out by the measurement of body fluid C3a levels rather than of body fluid C3b levels, since C3a remains in the fluid phase whereas C3b is rapidly cleared. C3a levels in a body fluid can be measured by methods well known in the art such as, for example, by using a commercially available C3a EIA kit, e.g., that sold by Quidel Corporation, San Diego, Calif., according to the manufacturer's specifications. Particularly specific antibodies capable of inhibiting complement such as an anti-C5 antibody produce essentially no reduction in body fluid C3a levels following complement activation when tested in such assays.

Certain antibodies of the disclosure will prevent the cleavage of C5 to form C5a and C5b, thus preventing the generation of the anaphylatoxic activity associated with C5a and preventing the assembly of the membrane attack complex associated with C5b. As discussed above, in a particular embodiment, these anti-C5 antibodies will not impair the opsonization function associated with the action of C3b.

A specific method of inhibiting complement activity is to use a monoclonal antibody which binds to complement C5 and prevents C5 from being cleaved. This prevents the formation of both C5a and C5b-9 while at the same time allowing the formation of C3a and C3b which are beneficial to the recipient. Such antibodies that are specific to human complement are known (U.S. Pat. No. 6,355,245). These antibodies disclosed in U.S. Pat. No. 6,355,245 include both a whole or full-length antibody (now named eculizumab) and a single-chain antibody (now named pexelizumab). A similar antibody against mouse C5 is called BB5.1 (Frei et al. (1987). Mol. Cell. Probes 1:141-149). BB5.1 was utilized in the experiments set forth below. Antibodies to inhibit complement activity need not be monoclonal antibodies. They can be, e.g., polyclonal antibodies. They may additionally be antibody fragments. An antibody fragment includes, but is not limited to, an Fab, Fab′, F(ab′)₂, a single-chain antibody, a domain antibody, and an Fv. Furthermore, it is well known by those of skill in the art that antibodies can be humanized (Jones et al. (1986). Nature 321:522-525), chimerized, or deimmunized. An antibody may also comprise an engineered Fc portion, such that the engineered Fc does not activate complement (WO 2005/007809). The antibodies to be used in the present invention may be any of these. Antibody analogs or mimics can also be used in the present invention, such as those described in U.S. Patent Application Publication No. 20050255548.

In certain embodiments, the disclosure provides a method of treating muscular dystrophy associated with dysferlin-deficiency in a mammal comprising administering to said mammal a therapeutically effective amount of a CD55 or soluble portion of CD55 or a mimic thereof. In certain embodiments, the mammal is a human. In certain embodiments, the CD55 or soluble portion of CD55 or mimic thereof inhibits the association of C4b and C3b. In particular embodiments, the CD55 or soluble portion of CD55 or mimic thereof blocks the formation of C3 or C5 convertases. CD55 activity can be measured by conventional methods, such as for example by measuring decay dissociation of the C4b2a enzyme, as described in Medof et al., J Exp Med. 160(5):1558-78 (1984).

### Therapeutic Agents: Antibodies and Other Agents

The disclosure provides various therapeutic agents. A therapeutic agent of the disclosure can be administered to a patient in need thereof as a single therapy. Alternatively, a therapeutic agent of the disclosure can be administered to a patient in need thereof in the form of combination therapy or adjuvant therapy. Such combination or adjuvant therapy further includes one or more other agents, such as other therapeutic agents (e.g., drugs or biologics) or nutritional agents (e.g., nutraceuticals or dietary supplements). As described herein, such combination therapy may include either simultaneous or sequential dosing or administration of the various agents as desired.

In certain embodiments, a therapeutic agent comprises an antibody or antibody fragment to C5 (or an anti-C5 antibody or fragment thereof). In certain embodiments, an anti-C5 antibody or fragment thereof binds C5 and inhibits C5 activity. For example, an anti-C5 antibody or its fragment of the disclosure binds C5 and blocks the cleavage of C5 into C5a and C5b. In certain embodiments, an anti-C5 antibody or fragment thereof binds C5 and results in a more rapid clearance of C5 from the plasma than will occur in the absence of the anti-C5 antibody or fragment thereof.

In certain embodiments, the therapeutic agent comprises an amino acid sequence of greater than 90% sequence identity to the amino acid sequence of a soluble portion of a naturally occurring CD55 protein. In certain embodiments, the therapeutic agent comprises a polynucleotide sequence of greater than 90% sequence identity to the nucleotide sequence of a naturally occurring CD55 mRNA. In another embodiment, the therapeutic agent comprises a polynucleotide sequence encoding an amino acid sequence of greater than 90% sequence identity to the amino acid sequence of a soluble portion of a naturally occurring CD55 protein. “Percent (%) nucleic acid or amino acid sequence identity” is defined as the percentage of nucleotides or amino acids in a candidate sequence that are identical with the nucleotides or amino acids in a reference sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Small molecule” as used herein, is meant to refer to an agent, which has a molecular weight of less than about 6 kD and most preferably less than about 2.5 kD. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J. Am. Chem. Soc. 120, 8565-8566, 1998). It is within the scope of this application that such a library may be used to screen for agents of the invention. There are numerous commercially available compound libraries, such as the Chembridge DIVERSet. Libraries are also available from academic investigators, such as the Diversity set from the NCI developmental therapeutics program. Rational drug design may also be employed. For example, the interaction interface of CD55 may be targeted when designing a compound.

Peptidomimetics can be compounds in which at least a portion of a subject polypeptide of the disclosure (such as for example, a polypeptide comprising an amino acid sequence of greater than 90% sequence identity to the amino acid sequence of a soluble portion of a naturally occurring CD55 protein) is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a nonpeptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements.

Nucleic acid analogs may include modified subject nucleic acid of the disclosure (such as for example, a nucleic acid comprising a polynucleotide sequence of greater than 90% sequence identity to the polynucleotide sequence of a naturally occurring CD55 gene). Various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

In specific embodiments, a therapeutic agent of the disclosure comprises an antibody or antibody fragment. Antibodies and fragments thereof may be made by any conventional method, such as those methods described herein.

Antibodies are found in multiple forms, e.g., IgA, IgG, IgM, etc. Additionally, antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

This invention provides fragments of anti-C5 antibodies, which may comprise a portion of an intact antibody, preferably the antigen-binding or variable region of the intact antibody. Examples of antibody fragments include Fab, Fab′, F(ab′)₂, and Fv fragments; diabodies; linear antibodies (Zapata et al., Protein Eng. 1995; 8(10): 1057-1062); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment of an antibody yields an F(ab′)₂ fragment that has two antigen-combining sites and is still capable of cross-linking antigen.

“Fv” usually refers to the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although likely at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

This disclosure also provides monoclonal anti-C5 antibodies. A monoclonal antibody can be obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are often synthesized by the hybridoma culture, uncontaminated by other immunoglobulins. Monoclonal antibodies may also be produced in transfected cells, such as CHO cells and NS0 cells. The modifier “monoclonal” indicates the character of the antibody as being obtained from a substantially homogeneous population of antibodies and does not require production of the antibody by any particular method. For example, the monoclonal antibodies to be used in accordance with the present disclosure may be made by the hybridoma method first described by Kohler et al., Nature 1975; 256:495, or may be made by recombinant DNA methods (see, e.g., U.S. Pat. Nos. 4,816,567 and 6,331,415). The “monoclonal antibodies” may also be isolated from phage antibody libraries using the techniques described in Clackson et al., Nature 1991; 352:624-628 and Marks et al., J. Mol. Biol. 1991; 222:581-597, for example.

General methods for the immunization of animals (in this case with C5 and/or C5b), isolation of antibody producing cells, fusion of such cells with immortal cells (e.g., myeloma cells) to generate hybridomas secreting monoclonal antibodies, screening of hybridoma supernatants for reactivity of secreted monoclonal antibodies with a desired antigen (in this case the immunogen or a molecule containing the immunogen), the preparation of quantities of such antibodies in hybridoma supernatants or ascites fluids, and for the purification and storage of such monoclonal antibodies, can be found in numerous publications. These include: Coligan et al., eds. Current Protocols In Immunology, John Wiley & Sons, New York, 1992; Harlow and Lane, Antibodies, A Laboratory Manual, Cold Spring Harbor Laboratory, New York, 1988; Liddell and Cryer, A Practical Guide To Monoclonal Antibodies, John Wiley & Sons, Chichester, West Sussex, England, 1991; Montz et al., Cellular Immunol. 127:337-351, 1990; Wurzner et al., Complement Inflamm. 8:328-340, 1991; and Mollnes et al., Scand. J. Immunol. 28:307-312, 1988.

A description of the preparation of a mouse anti-human-C5 monoclonal antibody with specific binding characteristics is presented in U.S. Patent Application Publication No. 20050226870. Wurzner et al., Complement Inflamm. 8:328-340, 1991, describe the preparation of other mouse anti-human-C5 monoclonal antibodies referred to as N19-8 and N20-9.

Other antibodies specifically contemplated are “oligoclonal” antibodies. As used herein, the term “oligoclonal” antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule (e.g., C5). In view of the assays and epitopes disclosed herein, those skilled in the art can generate or select antibodies or mixtures of antibodies that are applicable for an intended purpose and desired need.

In certain embodiments that include a humanized and/or chimeric antibody, one or more of the CDRs are derived from an anti-human C5 antibody. In a specific embodiment, all of the CDRs are derived from an anti-human C5 antibody. In another specific embodiment, the CDRs from more than one anti-human C5 antibody are mixed and matched in a chimeric antibody. For instance, a chimeric antibody may comprise a CDR1 from the light chain of a first anti-human C5 antibody combined with CDR2 and CDR3 from the light chain of a second anti-human C5 antibody, and the CDRs from the heavy chain may be derived from a third anti-human C5 antibody. Further, the framework regions may be derived from one of the same anti-human C5 antibodies, from one or more different antibodies, such as a human antibody, or from a humanized antibody. Human or humanized antibodies are specific for administration to human patients.

“Single-chain Fv” or “scFv” antibody fragments comprise the V_{H} and V_{L} domains of an antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv see Pluckthun in The Pharmacology of Monoclonal Antibodies, vol. 113, Rosenburg and Moore, eds. (Springer-Verlag: New York, 1994), pp. 269-315.

SMIPs are a class of single-chain peptide engineered to include a target binding region, effector domain (CH2 and CH3 domains). See, e.g., U.S. Patent Application Publication No. 20050238646. The target binding region may be derived from the variable region or CDRs of an antibody, e.g., an anti-C5 antibody of the invention. Alternatively, the target binding region is derived from a protein that binds C5.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In specific embodiments, the antibody will be purified to greater than 95% by weight of antibody as determined by the Lowry method, or greater than 99% by weight, to a degree that complies with applicable regulatory requirements for administration to human patients (e.g., substantially pyrogen-free), to a degree sufficient to obtain at least 15 residues of N-terminal or internal amino acid sequence by use of a spinning cup sequenator, or to homogeneity by SDS-PAGE under reducing or nonreducing conditions using Coomassie blue or, preferably, silver stain. Isolated antibody includes the antibody in situ within recombinant cells, since at least one component of the antibody's natural environment will not be present. Ordinarily, however, isolated antibody will be prepared by at least one purification step, for example, an affinity chromatography step, an ion (anion or cation) exchange chromatography step, or a hydrophobic interaction chromatography step.

It is well known that the binding to a molecule (or a pathogen) of antibodies with an Fc region assists in the processing and clearance of the molecule (or pathogen). The Fc portions of antibodies are recognized by specialized receptors expressed by immune effector cells. The Fc portions of IgG1 and IgG3 antibodies are recognized by Fc receptors present on the surface of phagocytic cells such as macrophages and neutrophils, which can thereby bind and engulf the molecules or pathogens coated with antibodies of these isotypes (C. A. Janeway et al., Immunobiology 5th edition, page 147, Garland Publishing (New York, 2001)).

In certain embodiments, single chain antibodies, and chimeric, humanized or primatized (CDR-grafted) antibodies, as well as chimeric or CDR-grafted single chain antibodies, comprising portions derived from different species, are also encompassed by the present disclosure as antigen-binding fragments of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. Nos. 4,816,567 and 6,331,415; U.S. Pat. No. 4,816,397; European Patent No. 0,120,694; WO 86/01533; European Patent No. 0,194,276 B1; U.S. Pat. No. 5,225,539; and European Patent No. 0,239,400 B1. See also, Newman et al., BioTechnology, 10: 1455-1460 (1992), regarding primatized antibody. See, e.g., Ladner et al., U.S. Pat. No. 4,946,778; and Bird et al., Science, 242: 423-426 (1988)), regarding single chain antibodies.

In addition, functional fragments of antibodies, including fragments of chimeric, humanized, primatized or single chain antibodies, can also be produced. Functional fragments of the subject antibodies retain at least one binding function and/or modulation function of the full-length antibody from which they are derived. Preferred functional fragments retain an antigen-binding function of a corresponding full-length antibody (such as for example, ability of anti-C5 antibody to bind C5).

### Pharmaceutical Formulations and Uses

Methods of administration of therapeutic agents, particularly antibody therapeutics, are well-known to those of skill in the art. The pharmaceutical formulations, dosage forms, and uses described below generally apply to antibody-based therapeutic agents, but are also useful and can be modified, where necessary, for making and using therapeutic agents of the disclosure that are not antibodies.

To achieve the desired therapeutic effect, the anti-C5 antibodies or CD55 peptidomimetics (or fragments thereof) can be administered in a variety of unit dosage forms. The dose will vary according to the particular antibody. For example, different antibodies may have different masses and/or affinities, and thus require different dosage levels. Antibodies prepared as Fab fragments will also require differing dosages than the equivalent intact immunoglobulins, as they are of considerably smaller mass than intact immunoglobulins, and thus require lower dosages to reach the same molar levels in the patient's blood. The dose will also vary depending on the manner of administration, the particular symptoms of the patient being treated, the overall health, condition, size, and age of the patient, and the judgment of the prescribing physician. Dosage levels of the antibodies for human subjects are generally between about 1 mg per kg and about 100 mg per kg per patient per treatment, and preferably between about 5 mg per kg and about 50 mg per kg per patient per treatment. In terms of plasma concentrations, the antibody concentrations are preferably in the range from about 25 μg/mL to about 500 μg/mL. However, greater amounts may be required for extreme cases and smaller amounts may be sufficient for milder cases.

Administration of the anti-C5 antibodies will generally be performed by an intravascular route, e.g., via intravenous infusion by injection. Other routes of administration may be used if desired but an intravenous route will be the most preferable. Formulations suitable for injection are found in Remington's Pharmaceutical Sciences, Mack Publishing Company, Philadelphia, Pa., 17th ed. (1985). Such formulations must be sterile and non-pyrogenic, and generally will include a pharmaceutically effective carrier, such as saline, buffered (e.g., phosphate buffered) saline, Hank's solution, Ringer's solution, dextrose/saline, glucose solutions, and the like. The formulations may contain pharmaceutically acceptable auxiliary substances as required, such as, tonicity adjusting agents, wetting agents, bactericidal agents, preservatives, stabilizers, and the like.

Administration of the antibodies capable of inhibiting complement such as an anti-C5 antibody will generally be performed by a parenteral route, typically via injection such as intra-articular or intravascular injection (e.g., intravenous infusion) or intramuscular injection. Other routes of administration, e.g., oral (p.o.), may be used if desired and practicable for the particular antibody capable of inhibiting complement to be administered. Antibodies capable of inhibiting complement such as an anti-C5 antibody can also be administered in a variety of unit dosage forms and their dosages will also vary with the size, potency, and in vivo half-life of the particular antibody capable of inhibiting complement being administered. Doses of antibodies capable of inhibiting complement such as an anti-C5 antibody will also vary depending on the manner of administration, the particular symptoms of the patient being treated, the overall health, condition, size, and age of the patient, and the judgment of the prescribing physician.

In certain embodiments, a typical therapeutic treatment includes a series of doses, which will usually be administered concurrently with the monitoring of clinical endpoints such as deposition of membrane attack complex (MAC) on nonnecrotic muscle fibers, age at reaching Hammersmith score of 30/40, age at becoming wheelchair bound, muscle pain or spasms, etc., with the dosage levels adjusted as needed to achieve the desired clinical outcome. In certain embodiments, treatment is administered in multiple dosages over at least a week. In certain embodiments, treatment is administered in multiple dosages over at least a month. In certain embodiments, treatment is administered in multiple dosages over at least a year. In certain embodiments, treatment is administered in multiple dosages over the remainder of the patient's life. In certain embodiments, treatment is administered chronically. “Chronically” as used herein, is meant to refer to administering the therapeutic for a period of at least 3 months, preferably for a period of at least 1 year, and more preferably for the duration of the disease in the patient.

The frequency of administration may also be adjusted according to various parameters. These include the clinical response, the plasma half-life of the antibody capable of inhibiting complement, and the levels of the antibody in a body fluid, such as, blood, plasma, serum, or synovial fluid. To guide adjustment of the frequency of administration, levels of the antibody capable of inhibiting complement in the body fluid may be monitored during the course of treatment.

Alternatively, for therapeutics or antibodies capable of inhibiting complement such as an anti-C5 antibody that affects C5b, levels of the cell-lysing ability of complement present in one or more of the patient's body fluids are monitored to determine if additional doses or higher or lower dosage levels are needed. Such doses are administered as required to maintain at least about a 25% reduction, and preferably about a 50% or greater reduction of the cell-lysing ability of complement present in blood, plasma, or serum. The cell-lysing ability can be measured as percent hemolysis in hemolytic assays of the types described herein. A 10% or 25% or 50% reduction in the cell-lysing ability of complement present in a body fluid after treatment with the antibody capable of inhibiting complement used in the practice of the invention means that the percent hemolysis after treatment is 90, 75, or 50 percent, respectively, of the percent hemolysis before treatment.

In yet another alternative, dosage parameters are adjusted as needed to achieve a substantial reduction of C5a levels in blood, plasma, or serum. As discussed above, C5a levels can be measured using the techniques described in Wurzner, et al., Complement Inflamm 8:328-340, 1991. Other protocols of administration can, of course, be used if desired as determined by the physician.

Administration of the therapeutics of the disclosure will generally be performed by a parenteral route, typically via injection such as intra-articular or intravascular injection (e.g., intravenous infusion) or intramuscular injection. Other routes of administration, e.g., oral (p.o.), may be used if desired and practicable for the particular antibody capable of inhibiting complement to be administered.

For the treatment of muscular dystrophy associated with dysferlin-deficiency by systemic administration of a therapeutic or antibody capable of inhibiting complement such as an anti-C5 antibody (as opposed to local administration), administration of a large initial dose is specific, i.e., a single initial dose sufficient to yield a substantial reduction, and more preferably an at least about 50% reduction, in the hemolytic activity of the patient's serum. Such a large initial dose is preferably followed by regularly repeated administration of tapered doses as needed to maintain substantial reductions of serum hemolytic titer. In another embodiment, the initial dose is given by both local and systemic routes, followed by repeated systemic administration of tapered doses as described above.

Formulations suitable for injection, p.o., and other routes of administration are well known in the art and may be found, for example, in Remington's Pharmaceutical Sciences, Mack Publishing Company, Philadelphia, Pa., 17th ed. (1985). Parenteral formulations must be sterile and non-pyrogenic, and generally will include a pharmaceutically effective carrier, such as saline, buffered (e.g., phosphate buffered) saline, Hank's solution, Ringer's solution, dextrose/saline, glucose solutions, and the like. These formulations may contain pharmaceutically acceptable auxiliary substances as required, such as, tonicity adjusting agents, wetting agents, bactericidal agents, preservatives, stabilizers, and the like.

Formulations particularly useful for antibody-based therapeutic agents are also described in U.S. Patent App. Publication Nos. 20030202972, 20040091490 and 20050158316. In certain embodiments, the liquid formulations of the invention are substantially free of surfactant and/or inorganic salts. In another specific embodiment, the liquid formulations have a pH ranging from about 5.0 to about 7.0. In yet another specific embodiment, the liquid formulations comprise histidine at a concentration ranging from about 1 mM to about 100 mM. In still another specific embodiment, the liquid formulations comprise histidine at a concentration ranging from 1 mM to 100 mM. It is also contemplated that the liquid formulations may further comprise one or more excipients such as a saccharide, an amino acid (e.g., arginine, lysine, and methionine) and a polyol. Additional descriptions and methods of preparing and analyzing liquid formulations can be found, for example, in PCT publications WO 03/106644, WO 04/066957, and WO 04/091658.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions of the invention.

In certain embodiments, formulations of the subject antibodies are pyrogen-free formulations which are substantially free of endotoxins and/or related pyrogenic substances. Endotoxins include toxins that are confined inside microorganisms and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin.

Formulations of the subject antibodies include those suitable for oral, dietary, topical, parenteral (e.g., intravenous, intraarterial, intramuscular, subcutaneous injection), ophthalmologic (e.g., topical or intraocular), inhalation (e.g., intrabronchial, intranasal or oral inhalation, intranasal drops), rectal, and/or intravaginal administration. Other suitable methods of administration can also include rechargeable or biodegradable devices and controlled release polymeric devices. Stents, in particular, may be coated with a controlled release polymer mixed with an agent of the invention. The pharmaceutical compositions of this disclosure can also be administered as part of a combinatorial therapy with other agents (either in the same formulation or in a separate formulation).

The amount of the formulation which will be therapeutically effective can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula:

Dose(mL)=[patient weight(kg)×dose level(mg/kg)/drug concentration (mg/mL)]

To achieve the desired reductions of body fluid parameters, such anti-C5 antibodies can be administered in a variety of unit dosage forms. The dose will vary according to the particular antibody. For example, different antibodies may have different masses and/or affinities, and thus require different dosage levels. Antibodies prepared as Fab′ fragments or single chain antibodies will also require differing dosages than the equivalent native immunoglobulins, as they are of considerably smaller mass than native immunoglobulins, and thus require lower dosages to reach the same molar levels in the patient's blood.

Other therapeutics of the disclosure can also be administered in a variety of unit dosage forms and their dosages will also vary with the size, potency, and in vivo half-life of the particular therapeutic being administered.

Doses of therapeutics of the disclosure will also vary depending on the manner of administration, the particular symptoms of the patient being treated, the overall health, condition, size, and age of the patient, and the judgment of the prescribing physician.

The formulations of the invention can be distributed as articles of manufacture comprising packaging material and a pharmaceutical agent which comprises, e.g., the antibody capable of inhibiting complement and a pharmaceutically acceptable carrier as appropriate to the mode of administration. The packaging material will include a label which indicates that the formulation is for use in the treatment of muscular dystrophy associated with dysferlin-deficiency.

Without intending to limit it in any manner, the present invention will be more fully described by the following examples. The methods and materials which are common to various of the examples are as follows.

### EXAMPLES

### Example 1

### DAF/CD55 is Down-Regulated in Dysferlin-Deficient Mice

The GeneChip Murine Genome U74Av2 array was used to compare the gene expression profiles of skeletal and cardiac muscles of SJL/J mice with dysferlin deficiency to those of C57BL/6 control mice. Analysis of gene expression in the nonpooled skeletal muscle of SJL/J vs. control mice revealed 291 differentially expressed genes at a threshold of p<0.001.

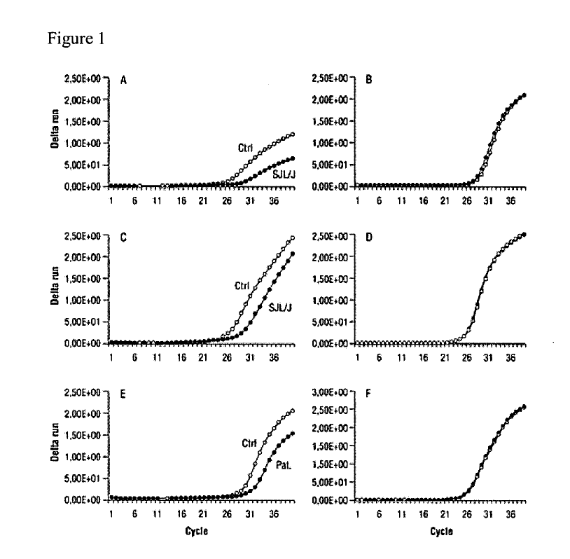

DAF1 was 5-fold down-regulated in skeletal muscle of SJL/J compared with skeletal muscle of C57/BL6 mice, with a significance of p=0.0000009. In contrast, in left cardiac ventricle, a mild 1.5-fold up-regulation was found. Similar results were observed with DAF2 (8.2-fold down-regulation in skeletal muscle, p=0.0027; 4-fold up-regulation in heart). Therefore, analyzed intraindividually, these two complement inhibitory factors, corresponding to human CD55, are significantly differentially expressed in skeletal muscle and heart. CD59, another well-described inhibitor of complement activation, was not differentially expressed (Table II). There was no significant difference in the expression of complement receptor 1, complement component factor H, or factor H-like 1 in skeletal muscles of dysferlin-deficient and control mice (Table II). The differential expression of DAF1 and DAF2 was validated by TaqMan RT-PCR and revealed a 4-fold down-regulation of DAF1 (p=0.005) and a 2-fold down-regulation of DAF2 (p=0.003; FIG. 1). The mild up-regulation of cardiac DAF was confirmed independently for the left and right ventricles (not shown).

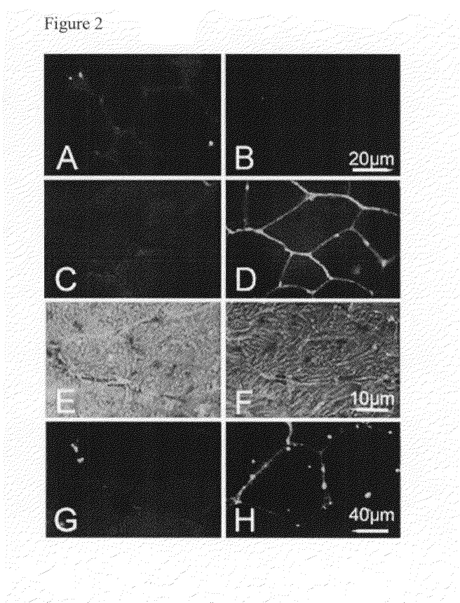

In accordance with the results obtained by microarray and TaqMan RT-PCR, the DAF/CD55 protein was absent by immunohistochemical staining of SJL/J quadriceps muscle, but was readily detectable on the sarcolemma of C57BL/6 control muscle (FIG. 2, A and D). A decrease in DAF/CD55 was found in SJL/J mice of all age groups (12, 16, 20, 28, and 32 wk; at least two mice per age group were tested), indicating that CD55 down-regulation is not merely a consequence of age and progressive dystrophic changes in muscle. Skeletal muscle tissues obtained from two additional dysferlin-deficient mouse strains (A/J and Dysf^{-l-}) at 16 wk of age also revealed the absence of DAF/CD55 (FIG. 2, B and C). Protein expression of DAF/CD55 in SJL/J myocardial tissue was not different from that in C57/BL6 control mice (FIG. 2, E and F).

### Example 2

### DAF/CD55 is Down-Regulated in LGMD2B Patients

Next, skeletal muscle from four patients with dysferlin-deficient muscular dystrophy (LGMD2B) was studied. The diagnosis of LGMD2B was confirmed by the absence of dysferlin in immunohistochemical staining, in Western blot analysis, and by genomic sequencing of DYSF (Table III). All patients had reduced sarcolemmal CD55 expression compared with normal skeletal muscle (FIG. 2, G and H). The degree of down-regulation of CD55 varied between patients, from trace staining to complete absence. Staining for CD46 and CD59, two other complement inhibitory molecules, was normal in all patients and controls (not shown).

The expression of DAF/CD55 in human skeletal muscle was also analyzed at the RNA level by TaqMan analysis. Compared with four control specimens from healthy individuals, DAF/CD55 mRNA in LGMD2B was 2.1-fold reduced (FIGS. 1, E and F).

### Example 3

### Dysferlin-Deficient Human Myotubes are Susceptible to Complement Attack

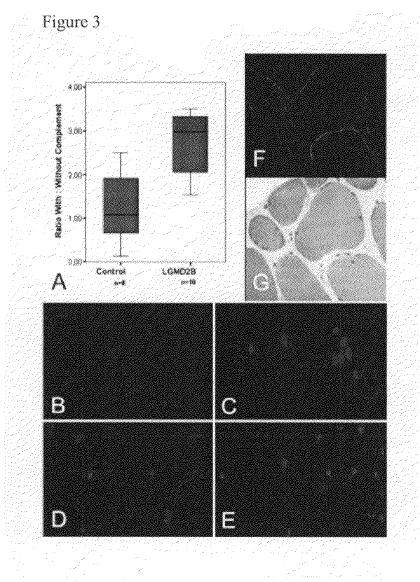

Functionally, the absence of CD55 should lead to an increased sensitivity to complement-mediated lysis. Human myotube cultures obtained from normal (n=2) and dysferlin-deficient human skeletal muscle (n=3; at least two independent experiments per patient) were established and exposed to complement-mediated lysis. Lysed and dead cells were identified by PI uptake. Normal human myotubes were resistant to complement-mediated lysis (FIGS. 3, A and B). This effect could be partially inhibited by preincubation with anti-CD55 Ab (FIG. 3C). On the contrary, myoblasts and myotubes obtained from patients with dysferlin deficiency were highly susceptible to complement attack (FIGS. 3, A and D). The percentage of lysed cells was not altered by the addition of anti-CD55 mAb (FIG. 3E).

The presence of the C5b9 MAC on the surface of nonnecrotic muscle fibers was demonstrated by immunohistochemistry in four of four muscle specimens obtained from dysferlin-deficient patients (FIGS. 3, F and G).

### Example 4

### DAF/CD55, Myostatin, and SMAD

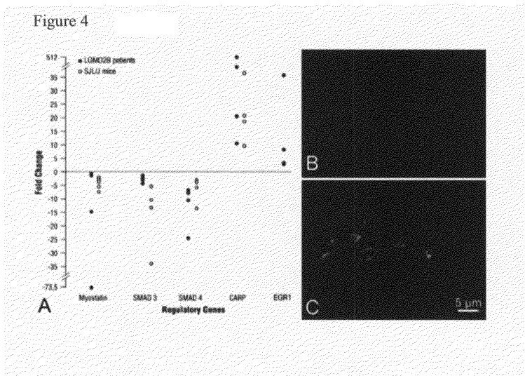

To elucidate possible regulatory mechanisms of DAF/CD55 in dysferlin deficiency, it was concluded that if DAF/CD55 down-regulation in dysferlin deficiency only plays a role in skeletal muscle, but not in heart, there should be genes that 1) are differentially expressed in dysferlin-deficient skeletal muscle and cardiac tissue and 2) regulate DAF/CD55. Indeed, within the microarray data obtained from dysferlin-deficient SJL/J mice, a small group of differentially expressed and potentially regulatory genes was identified: myostatin, SMAD2, SMAD3, SMAD4, cardiac ankyrin repeat protein (CARP), and early growth response 1 (EGR1). Therefore, the expression of these genes was quantified in skeletal and cardiac tissues by TaqMan RT-PCR in SJL/J mice and also in skeletal muscle from patients with dysferlin-deficient muscular dystrophy. In both mice and patients, compared with controls, myostatin, SMAD3, and SMAD4 were significantly down-regulated in skeletal muscle (FIG. 4A). In the heart, SMAD and myostatin were not differentially expressed in SJL/J and C57BL/6 mice (not shown). On the protein level, because of the availability of Abs, only phosphorylated SMAD2 was investigated, and it could be shown to also be markedly reduced in LGMD2B (FIG. 4B) compared with normal controls (FIG. 4C). CARP and EGR1 were strikingly up-regulated in skeletal muscle (FIG. 4A) and were reduced in heart (down-regulation of 2.5- and 4-fold in left and right ventricles, respectively).

### Example 5

### SMAD Binding Site in DAF/CD55 Promoter

To investigate whether any of these identified, differentially expressed, regulatory genes might influence DAF/CD55 expression, the DAF/CD55 promoter sequence (Ewulonu et al., 1991. Proc. Natl. Acad. Sci. USA 88:4675-4679) was analyzed for transcription factor binding sites using the MATInspector program (Genomatix) (Quandt et al., 1996. Comput. Appl. Biosci. 12:405-413). This analysis revealed a binding site for the SMAD complex, GTCTgggct (SEQ ID NO: 49) (Yingling et al., 1997. Mol. Cell. Biol. 17:7019-7028; Zawel et al., 1998. Mol. Cell 1:611-617; Dennler et al.,1998. EMBO J. 17:3091-3100), indicating that SMAD might influence DAF/CD55 expression. Among the 291 differentially expressed genes in skeletal muscle, other SMAD-regulated genes that are known to have significance for muscular dystrophies could not be identified.

### Example 6

### Anti-C5 Antibody Reduces Symptoms in Dysferlin-Deficient Mice

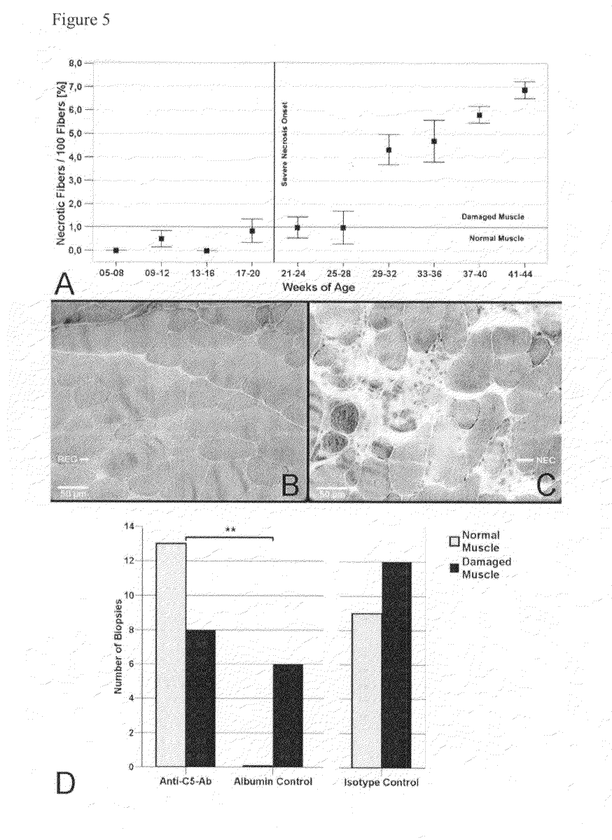

To investigate the efficacy of complement inhibition in vivo, dysferlin-deficient mice were treated with an anti-murine C5 antibody. SJL/J mice do not exhibit any clinical or histological signs of muscular dystrophy before week 20. Between week 22 and 26, there is a sharp increase in the number of necrotic fibers in muscle (FIG. 5A). Therefore, this time period was selected for anti-C5 treatment. The myopathological changes in SJL/J skeletal muscle were reduced by selective blockade of terminal complement with the anti-C5 antibody.

After four weeks of treatment with anti-C5 antibody, Gomori trichrome staining of quadriceps in SJL/J mice was analyzed to determine if the muscle samples had <1% necrotic fibers (“normal”) as compared to 1% or more necrotic fibers (“diseased”). Anti-C5 antibody treatment resulted in significantly fewer necrotic fibers than control animals treated with albumin (p<0.0005) (FIGS. 5B-D). Analyzing the difference between anti-C5 mAb treatment and isotype control IgG1 ab the effect size of dᵤ=−0.60 showed a significant medium-sized benefit of anti-C5 ab over IgG1. Therefore, anti-C5 treatment is effective in a mouse model of dysferlin-deficient muscular dystrophy.

### Example 7

### Materials and Methods

### Mice

Female SJL/J mice and C57BL/6 mice were purchased from Charles River Laboratories. The microarray experiments were performed in mice 32-34 wk of age. At this age, SJL/J mice showed marked histological signs of muscular dystrophy. Lymphomas were not detected. Muscle sections for immunohistochemistry were obtained from SJL/J mice at 12, 16, 20, 28, and 32 wk of age. For each age group, three mice were examined. Muscle sections from A/J and Dysf^{-l-} mice were obtained at 16 wk of age. All experiments were approved by local committees.

### Total RNA Preparation

RNA was extracted from mouse right quadriceps muscle, the left and right ventricles of mouse heart, and human skeletal muscle using TRIzol reagent (Invitrogen Life Technologies). Total RNA was treated by deoxyribonuclease I (Invitrogen Life Technologies) and was purified using the RNeasy Mini Kit (Qiagen).

### Microarray Experiments

Nonpooled microarray experiments were performed with cRNA prepared from quadriceps muscles and left ventricles of five SJL/J and five C57BL/6 mice using GeneChip Murine Genome U74Av2 (Affymetrix). Eight micrograms of RNA was transcribed in double-stranded cDNA using a cDNA Synthesis System (Roche). cRNA was produced by MEGAscript High Yield Transcription Kit (Ambion) and was labeled with biotin-11-CTP and biotin- 16-UTP nucleotides (PerkinElmer). Arrays were hybridized with 16 μg of fragmentized biotinylated cRNA at 45° C. and 60 rpm for 16 h in a GeneChip Hybridization Oven 640 (Affymetrix), washed and stained on a GeneChip Fluidics Station 400, and scanned in a GeneArray scanner 2500 (Affymetrix).

### Microarray Data Analysis

The resulting signals were processed using Affymetrix MicroArray Suite 5.0 software (MAS5.0) with a target intensity of 200. After standard data quality checks, the MAS5.0 expression signal values of each dataset were used for statistical analysis. Probe sets showing an absent call throughout all comparison groups were removed. A Nalimov test with a threshold of p<0.001 was used to exclude outliers. Student's t test (unpaired, two-tailed assumed unequal variance) was used to check the differences between two selected experimental groups.

### Quantitative Real-Time RT-PCR (TaqMan)

cDNA was synthesized from 5 μg of total RNA using PowerScript reverse transcriptase (BD Clontech) and an oligo(dT)₁₈ primer. Real-time PCR experiments were performed using TaqMan chemistry on an ABI PRISM 7700 Sequence Detection System (Applied Biosystems). Each reaction was performed in a singleplex format and contained TaqMan Universal PCR Master Mix (Applied Biosystems), 900 nM forward and reverse primers, and 200 nM TaqMan probe (BioTez). An annealing/extension temperature of 58° C. and 40 cycles were used. Primer/probe sets were designed using Primer Express 1.5 software (Applied Biosystems; Table I). For every sample, three independent runs in triplicate were performed, and the relative change in gene expression was quantified by the comparative threshold cycle method (Livak and Schmittgen. 2001. Methods 25:402-408). Unpaired two-tail unequal variance t test with a significance threshold of p<0.05 was used to compare the individual changes in threshold cycle values of the control and experimental group.

### Patients

Patients were followed in the Neuromuscular Unit of the Department of Neurology, Charité University Hospital (Berlin, Germany). Genomic sequencing of DYSF was performed if LGMD2B was suspected clinically and by immunohistochemistry and/or Western blotting. Patients included in this study gave their written informed consent. All studies were performed according to Declaration of Helsinki principles.

### Immunohistochemistry

Murine DAF was detected with polyclonal rat anti-mouse Ab (MDI) (Spiller et al., 1999. J. Immunol. Methods 224:51-60); human CD55 was detected with SM1141PS (Acris Antibodies). Anti-human C5b9 mAb (DakoCytomation) was applied for MAC detection. Anti-phospho-MADR2 mAb against phosphorylated SMAD2 was obtained from EMD Biosciences. Double-immunofluorescent staining for SMAD protein (FITC) and nuclear membrane protein lamin A/C (Novocastra; Cy3) were examined using a two-photon microscope (Leica).

### Complement Attack Assay

Myoblast/myotube cultures and complement attack assays were performed according to published protocols (Blau and Webster. 1981. Proc. Natl. Acad. Sci. USA 78:5623-5627; Gasque et al., 1996. J. Immunol. 156:3402-3411). Myoblasts were grown in SMG-Medium (Promo Cell) supplemented with Promo Cell Supplement Mix, gentamicin (40 μg/ml; Invitrogen Life Technologies), 2 mM glutamine, and 10% FCS. Myoblasts were transferred on 96-well plates and grown to near confluence. Differentiation into myotubes was induced with DMEM containing 2% heat-inactivated horse serum. For complement attack assays, wells were incubated in sexplicate for 30 min with normal human serum diluted ⅕ and 1/20 in Veronal buffer (Hollbom & Söhne) containing 1% BSA. Half the wells were preincubated with anti-human CD55 mAb (5 μg/ml). Propidium iodide (PI; 0.5 μg/ml) was added to assess killing. The total number of myotubes was compared with PI-positive cells using a fluorescent tissue culture microscope (Leica) and by FACS analysis.

Treatment with Anti-C5 Antibody

Female SJL/J mice were obtained from Charles River Laboratories, Sulzberg, Germany. Anti-mouse C5 monoclonal antibody (IgG1) and an isotype-matched control antibody were from Alexion Pharmaceuticals. In preliminary experiments, the optimal time period and primary outcome measure for complement-inhibitory treatment was evaluated in treatment groups of 3-6 animals. The percentage of necrotic fibers was defined as the primary outcome measure. Clinical symptoms as determined by the SHIRPA protocol (Rafael et al. Mamm Genome 2000; 11:725-728) were not sufficiently sensitive to monitor changes in this short period of time. Intraperitoneal injections of anti-murine C5 mAb (n=20), mouse IgG1control ab (n=20) or albumin (n=6) each at 40 mg/kg bodyweight were administered every 3 days for 4 weeks. Thereafter, the percentage of necrotic fibers in the quadriceps muscle was determined. From each biopsy (n=46), two independent observers counted at least 300 individual fibers. Data were analyzed by Chi-square test and, if group differences were undetected, by effect size analysis. Less than 1% necrotic fibers was interpreted as “normal”, whereas 1% or more were considered “diseased”.

TABLE I
Primer and probe sequences used for TaqMan RT-PCR

| Gene | | | | Ref. |
|---|---|---|---|---|
| Product | Forward Primer | Reverse Primer | Probe | Sequence |
| Mouse | | | | |
| PBGD | (SEQ ID NO.1) | (SEQ ID NO.9) | (SEQ ID NO.17) | NM_013551 |
| | GCACGATCCTGAAACTCTGC | TCCTTCCAGGTGCCTCAGAA | FAM-TCGCTGCATTGCTGAAAGGGCT-TAMRA | |
| DAF1 | (SEQ ID NO.2) | (SEQ ID NO.15) | (SEQ ID NO.18) | NM_010016 |
| | GTACAGGAACCCCCTCAACG | TGAGGAGTTGGTTGGTCTCC | FAM-CAGAAACCCACAACAGAAAGTGTTCCAAA | |
| | | | T-TAMRA | |
| DAF2 | (SEQ ID NO.3) | (SEQ ID NO.11) | (SEQ ID NO.19) | NM_007827 |
| | ACAGGAATCCCCTCAACGC | CTGAGGAGTTGGTTGGTCTCC | FAM-CAGAAACCCACAACAGAAAGTGTTCCAAAT | |
| | | | CC-TAMRA | |
| Myostatin | (SEQ ID NO.4) | (SEQ ID NO.12) | (SEQ ID NO.20) | NM_010834 |
| | AGGTGACAGACAGACCCAAGA | GATTCCGTGGAGTGCTCATC | FAM-TCCCGGAGAGACTTTGGGCTTGACTG- | |
| | | | TAMRA | |
| CARP | (SEQ ID N0.5) | (SEQ ID NO.13) | (SEQ ID NO.21) | NM_013468 |
| | GGACTGGTCATTACGAGTGCG | CCTTGGCATTGAGATCAGCC | FAM-TGAGCACCTCATCGCCTGCG-TAMRA | |
| SMAD2 | (SEQ ID NO.6) | (SEQ ID NO.14) | (SEQ ID NO.22) | NM_010754 |
| | CCCATTCCTGTTCTGGTTCA | AGCCAGCAGTGCAACTTTTT | FAM-AGCAGTACAGCAGAATGACGTCGTGC-TAMRA | |
| SMAD3 | (SEQ ID NO.7) | (SEQ ID NO.15) | (SEQ ID NO.23) | NM_016769 |
| | GGGCCTACTGTCCAATGTCA | CCCAATGTGTCGCCTTGTA | FAM-CCGGAATGCAGCCGTGGAAC-TAMRA | |
| SMAD4 | (SEQ ID NO.8) | (SEQ ID NO.16) | (SEQ ID NO.24) | NM_008540 |
| | CTGGACAGAGAAGCTGGCC | ACGCGCTTGGGTAGATCTT | FAM-AGCACCTGGCGACGCTGTTCA-TAMRA | |
| Human | | | | |
| β2MG | (SEQ ID NO.25) | (SEQ ID NO.33) | (SEQ ID NO.41) | NM_004048 |
| | ACTGAAAAAGATGAGTATGCCTGC | CATCTTCAAACCTCCATGATGCT | FAM-TGAACCATGTGACTTTGTCACAGCCCA- | |
| | | | TAMRA | |
| DAF/CD55 | (SEQ ID NO.26) | (SEQ ID NO.34) | (SEQ ID NO.42) | NM_000574 |
| | AAGTAATCTTTGGCTGTAAGGCA | TTCACCAGCATGTTTTACCTTTAA | FAM-TTTCATCTTTCCTTCGGGTTGGCAAA- | |
| | | | TAMRA | |
| Myostatin | (SEQ ID NO.27) | (SEQ ID NO.35) | (SEQ ID NO.43) | NM_005259 |
| | TGCTGTAACCTTCCCAGGAC | GGTGTGTCTGTTACCTTGACCTC | FAM-AGGAGAAGATGGGCTGAATCCGTTTTT- | |
| | | | TAMRA | |
| EGR1 | (SEQ ID NO.28) | (SEQ ID NO.36) | (SEQ ID NO.44) | NM_001964 |
| | TTCACGTCTTGGTGCCTTTT | CCCTCACAATTGCACATGTCA | FAM-TGATGCGCCTTGCTGATGGC-TAMRA | |
| CARP | (SEQ ID NO.29) | (SEQ ID NO.37) | (SEQ ID NO.45) | NM_014391 |
| | AAGTTGCTCAGCACAGCGCT | TGCTCCGCGCACTCATAGT | FAM-CATGTGGCGGTGAGGACTGGC-TAMRA | |
| SMAD2 | (SEQ ID NO.31) | (SEQ ID NO.38) | (SEQ ID NO.46) | NM_005901 |
| | TGTTTTAGTGCCCTGCTGC | GCTCACAAGATGGGTAGTGGA | FAM-CTTCCAGACTTTGTGCTGTCCAGTAATTAT | |
| | | | GTC-TAMRA | |
| SMAD3 | (SEQ ID NO.31) | (SEQ ID NO.35) | (SEQ ID NO.47) | NM_005902 |
| | GGGCACAGCCAGTTCTGAA | TTGGTGTTTCTGGATGCTGA | FAM-TTGGTGGAGGGTGTAGTGGCTTTTTGG- | |
| | | | TAMRA | |
| SMAD4 | (SEQ ID NO.32) | (SEQ ID NO.41) | (SEQ ID NO.48) | NM_005359 |
| | CAGCCGTGGCAGGAAAC | GCTGACAGACTGATAGCTGGAGC | FAM-TCCCTGGCCCAGGATCAGTAGGTGGA- | |
| | | | TAMRA | |

TABLE II
mRNA expression of regulatory proteins of the complement system in SJL/J mice

| | Skeletal | | | Heart | | | |
|---|---|---|---|---|---|---|---|
| | Muscle | | | (LV)^{a} | | | |
| | Fold | | | Fold | | | Accession |
| | change | Direction^{a} | p Value | change | Direction | p Value | No. |
| Decay | −5.2 | Down | 0.0000009 | 1.5 | Up | 0.000003 | NM_010016 |
| accelerating | | | | | | | |
| factor 1 | | | | | | | |
| Decay | −8.2 | Down | 0.0027 | 4 | Up | 0.0018 | NM_007827 |
| accelerating | | | | | | | |
| factor 2 | | | | | | | |
| C1 inhibitor | 1.3 | NC | 0.11 | 1.8 | Up | 0.005 | NM_009776 |
| Complement | 1.5 | NC | 0.08 | 1.6 | Up | 0.0012 | NM_010740 |
| receptor 1 | | | | | | | |
| Complement | 1.9 | NC | 0.23 | 2.1 | Up | 0.00002 | NM_009888 |
| component | | | | | | | |
| factor h | | | | | | | |
| Complement | 1.3 | NC | 0.06 | 2.1 | Up | 0.0046 | NM_015780 |
| component | | | | | | | |
| factor H-like | | | | | | | |
| 1 | | | | | | | |
| CD59a Ag | 1.7 | NC | 0.21 | −0.9 | NC | 0.34 | NM_007652 |
| (protectin) | | | | | | | |

^{a}LV, Left ventricle; NC, not significantly changed.

TABLE III
Patients with limb girdle musclular dystrophy 2B^{a}

| Patient | Gene | Age | Sex | Alleles | Mutation |
|---|---|---|---|---|---|
| 1 | DYSF | 52 | M | Homozygous | c.4022T > C |
| 2 | DYSF | 43 | F | Heterozygous | c.855 + 1delG |
| | | | | Heterozygous | c.895G > A |
| 3 | DYSF | 39 | M | Heterozygous | c.855 + 1delG |
| | | | | Heterozygous | c.895G > A |
| 4 | DYSF | 32 | F | Heterozygous | c.1448C > A |
| | | | | Heterozygous | c.6350T > A |

^{a}The mutations are submitted to the Leiden Muscular Dystrophy Database (http://dmd.nl). The MIAME-compliant microarray data are available at (www.ncbi.nlm.nih.gov/geo) under accession no. GSE2507.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method of treating muscular dystrophy associated with dysferlin-deficiency in a mammal comprising administering to said mammal a therapeutically effective amount of a complement inhibitor.

2. The method of claim 1, wherein said inhibitor inhibits terminal complement activity.

3. The method of claim 1, wherein said inhibitor inhibits C5a activity.

4. The method of claim 1, wherein said inhibitor inhibits cleavage of C5.

5. The method of claim 1, wherein said mammal is a human.

6. The method of claim 1 wherein said complement inhibitor is selected from: a polypeptide, a polypeptide analog, a peptidomimetic, an antibody, a nucleic acid, an RNAi construct, a nucleic acid analog, and a small molecule.

7. The method of claim 1, wherein said inhibitor is an antibody or an antibody fragment.

8. The method of claim 7, wherein said antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, and an F(ab′)₂.

9. The method of claim 1, wherein said complement inhibitor is administered chronically to said mammal.

10. The method of claim 1, wherein said complement inhibitor is administered systemically to said mammal.

11. The method of claim 1, wherein said complement inhibitor is administered locally to said mammal.

12. The method of claim 1, wherein said method does one or more of the following: slows muscles from weakening, slows the development of deformities, or delays loss of muscle function.

13. A method of treating muscular dystrophy associated with dysferlin-deficiency in a mammal comprising administering to said mammal a therapeutically effective amount of: a) a protein comprising an amino acid sequence of greater than 90% sequence identity to the amino acid sequence of a soluble portion of a naturally occurring CD55 protein, b) a nucleic acid comprising a polynucleotide sequence of greater than 90% sequence identity to the nucleotide sequence of a naturally occurring CD55 mRNA, or c) a nucleic acid encoding a protein comprising an amino acid sequence of greater than 90% sequence identity to the amino acid sequence of a soluble portion of a naturally occurring CD55 protein.

14. A method of reducing necrosis of muscle fibers in muscular dystrophy associated with dysferlin-deficiency in a mammal comprising administering to said mammal a therapeutically effective amount of complement inhibitor.

15. The method of claim 14, wherein said inhibitor inhibits terminal complement activity.

16. The method of claim 14, wherein said inhibitor inhibits C5a activity.

17. The method of claim 14, wherein said inhibitor inhibits cleavage of C5.

18. The method of claim 14, wherein said mammal is a human.

19. The method of claim 14, wherein said complement inhibitor is selected from:
a polypeptide, a polypeptide analog, a peptidomimetic, an antibody, a nucleic acid, an RNAi construct, a nucleic acid analog, and a small molecule.

20. The method of claim 14, wherein said inhibitor is an antibody or antibody fragment.

21. The method of claim 20, wherein said antibody or antibody fragment is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fab, an Fab′, and an F(ab′)₂.

22. The method of claim 14, wherein said complement inhibitor is administered chronically to said mammal.

23. The method of claim 14, wherein said complement inhibitor is administered systemically to said mammal.

24. The method of claim 14, wherein said complement inhibitor is administered locally to said mammal.

25. A method of reducing necrosis of muscle fibers in muscular dystrophy associated with dysferlin-deficiency in a mammal comprising administering to said mammal a therapeutically effective amount of: a) a protein comprising an amino acid sequence of greater than 90% sequence identity to the amino acid sequence of a soluble portion of a naturally occurring CD55 protein, b) a nucleic acid comprising a polynucleotide sequence of greater than 90% sequence identity to the nucleotide sequence of a naturally occurring CD55 mRNA, or c) a nucleic acid encoding a protein comprising an amino acid sequence of greater than 90% sequence identity to the amino acid sequence of a soluble portion of a naturally occurring CD55 protein.


SEQUENCE LISTING (49 sequences)

SEQ ID NO: 1 - DNA, 20 bp, Mouse
gcacgatcctgaaactctgc

SEQ ID NO: 2 - DNA, 20 bp, Mouse
gtacaggaaccccctcaacg

SEQ ID NO: 3 - DNA, 19 bp, Mouse
acaggaatcccctcaacgc

SEQ ID NO: 4 - DNA, 21 bp, Mouse
aggtgacagacacacccaaga

SEQ ID NO: 5 - DNA, 21 bp, Mouse
ggactggtcattacgagtgcg

SEQ ID NO: 6 - DNA, 20 bp, Mouse
cccattcctgttctggttca

SEQ ID NO: 7 - DNA, 20 bp, Mouse
gggcctactgtccaatgtca

SEQ ID NO: 8 - DNA, 19 bp, Mouse
ctggacagagaagctggcc

SEQ ID NO: 9 - DNA, 20 bp, Mouse
tccttccaggtgcctcagaa

SEQ ID NO: 10 - DNA, 20 bp, Mouse
tgaggagttggttggtctcc

SEQ ID NO: 11 - DNA, 21 bp, Mouse
ctgaggagttggttggtctcc

SEQ ID NO: 12 - DNA, 20 bp, Mouse
gattccgtggagtgctcatc

SEQ ID NO: 13 - DNA, 20 bp, Mouse
ccttggcattgagatcagcc

SEQ ID NO: 14 - DNA, 20 bp, Mouse
agccagcagtgcaacttttt

SEQ ID NO: 15 - DNA, 19 bp, Mouse
cccaatgtgtcgccttgta

SEQ ID NO: 16 - DNA, 19 bp, Mouse
acgcgcttgggtagatctt

SEQ ID NO: 17 - DNA, 22 bp, Mouse
  modified_base 1: This has a FAM dye attached
tcgctgcattgctgaaagggct

SEQ ID NO: 18 - DNA, 30 bp, Mouse
  modified_base 1: This has a FAM dye attached
cagaaacccacaacagaaagtgttccaaat

SEQ ID NO: 19 - DNA, 32 bp, Mouse
  modified_base 1: This has a FAM dye attached
cagaaacccacaacagaaagtgttccaaatcc

SEQ ID NO: 20 - DNA, 26 bp, Mouse
  modified_base 1: This has a FAM dye attached
tcccggagagactttgggcttgactg

SEQ ID NO: 21 - DNA, 20 bp, Mouse
  modified_base 1: This has a FAM dye attached
tgagcacctcatcgcctgcg

SEQ ID NO: 22 - DNA, 26 bp, Mouse
  modified_base 1: This has a FAM dye attached
agcagtacagcagaatgacgtcgtgc

SEQ ID NO: 23 - DNA, 20 bp, Mouse
  modified_base 1: This has a FAM dye attached
ccggaatgcagccgtggaac

SEQ ID NO: 24 - DNA, 21 bp, Mouse
  modified_base 1: This has a FAM dye attached
agcacctggcgacgctgttca

SEQ ID NO: 25 - DNA, 24 bp, Mouse
actgaaaaagatgagtatgcctgc

SEQ ID NO: 26 - DNA, 23 bp, Mouse
aagtaatctttggctgtaaggca

SEQ ID NO: 27 - DNA, 20 bp, Mouse
tgctgtaaccttcccaggac

SEQ ID NO: 28 - DNA, 20 bp, Mouse
ttcacgtcttggtgcctttt

SEQ ID NO: 29 - DNA, 20 bp, Mouse
aagttgctcagcacagcgct

SEQ ID NO: 30 - DNA, 19 bp, Mouse
tgttttagtgccctgctgc

SEQ ID NO: 31 - DNA, 19 bp, Mouse
gggcacagccagttctgaa

SEQ ID NO: 32 - DNA, 17 bp, Mouse
cagccgtggcaggaaac

SEQ ID NO: 33 - DNA, 23 bp, Mouse
catcttcaaacctccatgatgct

SEQ ID NO: 34 - DNA, 24 bp, Mouse
ttcaccagcatgttttacctttaa

SEQ ID NO: 35 - DNA, 23 bp, Mouse
ggtgtgtctgttaccttgacctc

SEQ ID NO: 36 - DNA, 21 bp, Mouse
ccctcacaattgcacatgtca

SEQ ID NO: 37 - DNA, 19 bp, Mouse
tgctccgcgcactcatagt

SEQ ID NO: 38 - DNA, 21 bp, Mouse
gctcacaagatgggtagtgga

SEQ ID NO: 39 - DNA, 20 bp, Mouse
ttggtgtttctggatgctga

SEQ ID NO: 40 - DNA, 23 bp, Mouse
gctgacagactgatagctggagc

SEQ ID NO: 41 - DNA, 27 bp, Mouse
  modified_base 1: This has a FAM dye attached
tgaaccatgtgactttgtcacagccca

SEQ ID NO: 42 - DNA, 26 bp, Mouse
  modified_base 1: This has a FAM dye attached
tttcatctttccttcgggttggcaaa

SEQ ID NO: 43 - DNA, 27 bp, Mouse
  modified_base 1: This has a FAM dye attached
aggagaagatgggctgaatccgttttt

SEQ ID NO: 44 - DNA, 20 bp, Mouse
  modified_base 1: This has a FAM dye attached
tgatgcgccttgctgatggc

SEQ ID NO: 45 - DNA, 21 bp, Mouse
  modified_base 1: This has a FAM dye attached
catgtggcggtgaggactggc

SEQ ID NO: 46 - DNA, 33 bp, Mouse
  modified_base 1: This has a FAM dye attached
cttccagactttgtgctgtccagtaattatgtc

SEQ ID NO: 47 - DNA, 27 bp, Mouse
  modified_base 1: This has a FAM dye attached
ttggtggagggtgtagtggctttttgg

SEQ ID NO: 48 - DNA, 26 bp, Mouse
  modified_base 1: This has a FAM dye attached
tccctggcccaggatcagtaggtgga

SEQ ID NO: 49 - DNA, 9 bp, Mouse
gtctgggct